(19) United States
(12) Patent Application Publication
(10) Pub. No.: US 2024/0050205 A1
(43) Pub. Date: Feb. 15, 2024

(54) METHOD OF SETTING CUSTOMIZABLE TEETH COLOR AND THREE-DIMENSIONAL PRINTING

(71) Applicants: 3D INDUSTRIAL IMAGING CO., LTD., Seoul (KR); SEOUL NATIONAL UNIVERSITY R&DB FOUNDATION, Seoul (KR)

(72) Inventors: Hyungbum KIM, Suwon-si (KR); Kyunyeon KIM, Anyang-si (KR); Jinsoo AHN, Seoul (KR); Sung-won JU, Seoul (KR)

(21) Appl. No.: 18/258,081

(22) PCT Filed: Dec. 17, 2021
(86) PCT No.: PCT/KR2021/019286

§ 371 Date: Jun. 16, 2023

(30) Foreign Application Priority Data
Dec. 18, 2020 (KR) 10-2020-0179065

(51) Int. Cl. A61C 13/08; A61C 13/00

(52) U.S. Cl. CPC A61C 13/082; A61C 13/0019; B33Y 10/00

(57) ABSTRACT

According to one embodiment of the present invention, there is provided a method for setting a customized tooth color, comprising: a first step of generating a patient's tooth color data; a second step of setting tooth color data for each section of artificial tooth based on the patient's tooth color data; a third step of performing an aesthetic prediction display based on the tooth color data for each section of artificial tooth, and correcting errors by comparing the artificial tooth displayed by the aesthetic prediction display with the patient's tooth color data; and a fourth step of determining artificial tooth color data by reflecting error correction.

### TECHNICAL FIELD

The present invention relates to methods for setting customized tooth color and 3D printing thereof.

### BACKGROUND ART

In recent years, various types of dental materials such as implants have been used. The aesthetic aspects of the dental materials, such as implants or dentures, are just as important as the functional aspects, which ensure the performance of a chewing action in place of natural teeth. In particular, recently, since materials such as laminates that emphasize aesthetic aspects rather than functional aspects are widely used, it can be said that the importance of aesthetics is greater than ever.

However, artificial teeth are currently manufactured by dental technicians by visually contrasting a standard color chart and an image of the patient's teeth, but this method has a problem in that the result may vary depending on the skill level of the dental technician. In addition, it is difficult even for a skilled dental technician to match the color of teeth, which are different for each individual, based on a fixed standard color chart. Therefore, the current manufacturing of artificial teeth has limitations in enhancing aesthetics in that it does not reflect individual characteristics.

### DISCLOSURE

### Technical Problem

It is an object of the present invention to provide an artificial tooth similar to a patient's actual tooth in consideration of the patient's tooth color and brightness.

### Technical Solution

According to one embodiment of the present invention, there is provided a method for setting a customized tooth color, including:
a first step of generating a patient's tooth color data;
a second step of setting tooth color data for each section of artificial tooth based on the patient's tooth color data;
a third step of performing an aesthetic prediction display based on the tooth color data for each section of artificial tooth, and correcting errors by comparing the artificial tooth displayed by the aesthetic prediction display with the patient's tooth color data; and
a fourth step of determining artificial tooth color data by reflecting error correction.

According to one embodiment of the present invention, there is provided the method for setting a customized tooth color, wherein the first step includes generating the patient's tooth color data by measuring colors in each of a first region, a second region, and a third region provided sequentially from the root of the tooth along the height direction of the tooth.

According to one embodiment of the present invention, there is provided the method for setting a customized tooth color, wherein the first step includes generating the patient's tooth color data by measuring color in an arbitrary region within the tooth and calculating a color deviation from the root of the tooth along the height direction.

According to one embodiment of the present invention, there is provided the method for setting a customized tooth color, wherein the tooth color data is represented by the b* value of the CIE L*a*b* coordinate system.

According to one embodiment of the present invention, there is provided the method for setting a customized tooth color, wherein the second step calculates L and a* values based on the b* value of the patient's tooth color data received in the first step and the preset tooth image model to generate tooth color data for each section of the artificial tooth.

According to one embodiment of the present invention, there is provided the method for setting a customized tooth color, wherein the tooth color data for each section of the artificial tooth includes section data each consisting of the start and end values of L and a*.

According to one embodiment of the present invention, there is provided the method for setting a customized tooth color, wherein the aesthetic prediction display in the third step shows a colored tooth image based on the tooth color data for each section of the artificial tooth, and the tooth image is overlaid with a glossy image.

According to one embodiment of the present invention, there is provided the method for setting a customized tooth color, wherein the method further performs calculating the mixing ratio of materials for printing an artificial tooth after the fourth step.

According to one embodiment of the present invention, there is provided the method for setting a customized tooth color, wherein the step of calculating the mixing ratio of the materials is to determine the mixing ratio of at least two or more materials having different colors.

According to one embodiment of the present invention, there is provided a computer program stored on a recording medium that can be interlocked with a computer device such that a method for setting a customized tooth color can be performed, wherein the method includes:
a first step of generating a patient's tooth color data;
a second step of setting tooth color data for each section of artificial tooth based on the patient's tooth color data;
a third step of performing an aesthetic prediction display based on the tooth color data for each section of artificial tooth, and correcting errors by comparing the artificial tooth displayed by the aesthetic prediction display with the patient's tooth color data; and
a fourth step of determining artificial tooth color data by reflecting error correction.

According to one embodiment of the present invention, there is provided a method for printing an artificial tooth, including: three-dimensionally printing an artificial tooth using at least two or more materials having different colors, and performing a method for setting a customized tooth color in order to determine the mixing ratio of the at least two or more materials, wherein the method includes:
a first step of generating a patient's tooth color data;
a second step of setting tooth color data for each section of artificial tooth based on the patient's tooth color data;
a third step of performing an aesthetic prediction display based on the tooth color data for each section of artificial tooth, and correcting errors by comparing the artificial tooth displayed by the aesthetic prediction display with the patient's tooth color data; and
a fourth step of determining artificial tooth color data by reflecting error correction.

### Advantageous Effects

According to one embodiment of the present invention, an artificial tooth similar to a patient's actual tooth may be provided using color and brightness coordination, and accordingly, aesthetic property may be improved during an artificial tooth procedure.

In addition, according to one embodiment of the present invention, an artificial tooth similar to a patient's actual tooth can be easily provided through 3D printing.

### BRIEF DESCRIPTION OF THE DRAWINGS

FIG. 1 is a flowchart showing a method for performing a customized tooth color setting according to one embodiment of the present invention.

FIG. 2 is a flowchart showing a method for performing 3D printing after setting the customized tooth color according to one embodiment of the present invention.

FIG. 3 is a block diagram showing a customized tooth color implementation system according to one embodiment of the present invention.

FIG. 4 is a graph and image data showing a color setting method according to one embodiment of the present invention.

FIG. 5 is a graph showing a color setting method according to one embodiment of the present invention.

FIG. 6 is a graph showing preparation steps for manufacturing artificial teeth according to one embodiment of the present invention.

FIG. 7 is an image showing an artificial tooth manufactured according to one embodiment of the present invention.

FIG. 8 is a flowchart showing in detail a method for setting a customized tooth color according to one embodiment of the present invention.

FIG. 9 is an actual implementation screen of a method for setting a customized tooth color according to one embodiment of the present invention.

### DETAILED DESCRIPTION OF PREFERRED EMBODIMENTS

The present invention can be variously changed and may have a number of forms, and thus specific embodiments will be illustrated in the drawings and described herein in detail. However, this is not intended to limit the invention to the specific form disclosed, but it should be understood that the present invention include all changes, equivalents and substitutes included in the spirit and scope of the present invention.

Upon describing the drawings, similar reference numerals are used for similar elements. In the accompanying drawings, the dimensions of the structures may be enlarged than the actual dimensions for clarity of the invention. Although the terms first, second, etc., may be used herein to describe various elements, these elements should not be limited by these terms. These terms are only used to distinguish one element from another element. For example, a first element could be termed a second element, and, similarly, a second element could be termed a first element, without departing from the scope of the present invention. Further, the singular forms are intended to include the plural forms as well, unless the context clearly indicates otherwise.

In the present application, it will be appreciated that terms “including” and “having” are intended to designate the existence of stated features, numbers, steps, operations, constituent elements, and components described in the specification or a combination thereof, and do not exclude the possibility of the existence or addition of one or more other features, numbers, steps, operations, constituent elements, and components, or a combination thereof in advance. It will be understood that when an element such as a layer, film, region, or substrate is placed “on” or “above” another element, it indicates not only a case where the element is placed “directly on” or “just above” the other element but also a case where a further element is interposed between the element and the other element. Further, in the present specification, when an element such as a layer, film, region, or substrate is placed on another element, the formed direction is not limited to an upper direction, but a side and a lower direction may also be included. On the contrary, it will be understood that when an element such as a layer, film, region, or substrate is placed “beneath” or “below” another element, it indicates not only a case where the element is placed “directly beneath” or “just below” the other element but also a case where a further element is interposed between the element and the other element.

In the present specification, the terms “front side” and “back side” are used as relative concepts to facilitate the understanding of the inventive concept. Therefore, the “front side” and “back side” do not designate a specific direction, position, or constituent element and may be interchangeably used. For example, “front side” may be interpreted as “back side”, and “back side” may be interpreted as “front side”. Therefore, “front side” may be represented as “first side”, and “back side” may be represented as “second side”, while “back side” may be represented as “first side” and “front side” may be represented as “second side”. However, “front side” and “back side” are not mixed with each other in one embodiment.

According to one embodiment of the present invention, while generating color data for manufacturing artificial teeth from patient's tooth color data, it is possible to manufacture artificial teeth very similar to actual teeth by dividing teeth into several sections and adjusting color data for each section. In addition, as the color is determined for each section, it is suitable to manufacture by printing artificial teeth using a 3D printer.

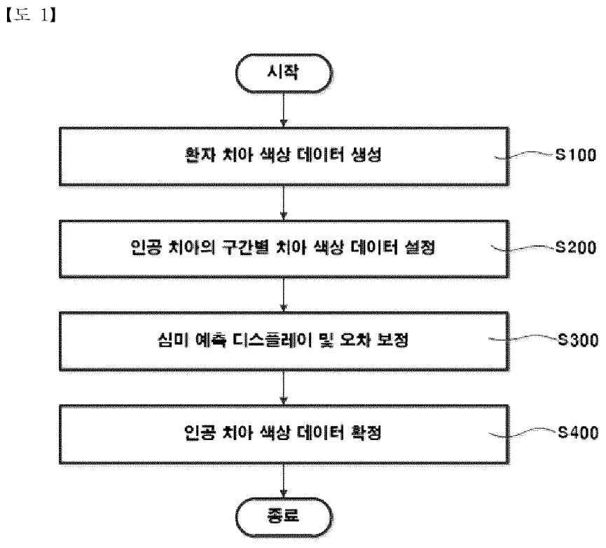

FIG. 1 is a flowchart showing a method for performing a customized tooth color setting according to one embodiment of the present invention.

With reference to FIG. 1, there is provided a method for setting a customized tooth color, including: a first step (S100) of generating a patient's tooth color data, a second step (S200) of setting tooth color data for each section of artificial tooth based on the patient's tooth color data, a third step (S300) of performing an aesthetic prediction display based on the tooth color data for each section of artificial tooth, and correcting errors by comparing the artificial tooth displayed by the aesthetic prediction display with the patient's tooth color data, and a fourth step (S400) of determining artificial tooth color data by reflecting error correction.

The first step (S100) is a step of generating a patient's tooth color data by examining the patient's teeth. In particular, the patient refers to a target to be subjected to an artificial tooth attachment procedure. The first step (S100) includes a step of examining the color of the patient's teeth by a method such as photographing the patient's teeth. For example, in the first step (S100), the patient's teeth may be photographed, or color measurement may be performed using a spectrophotometer, etc.

The patient's tooth color data generated in the first step (S100) may include color coordinate values, brightness values, etc., displayed according to a color coordinate system. For example, if the color coordinate system used by the customized tooth color setting system is the Lightness-Chroma-hue (LCh) coordinate system, the patient's tooth color data includes C coordinate values representing color, h coordinate values, and L coordinate values representing brightness. If the color coordinate system is the CIE L*a*b* coordinate system, the patient's tooth color data may include a* coordinate values representing color, b* coordinate values, and L* coordinate values representing brightness. However, the above-described color coordinate system is only exemplary, and other color coordinate systems may be used if necessary, and in this case, the form of patient's tooth color data may also vary.

The first step (S100) may be performed by dividing the tooth to be measured into several sections, measuring the color of the tooth for each section, and then setting color coordinate values for each section. In particular, the sections dividing the tooth to be measured may be determined according to the resolution for measuring the color of the patient's tooth. For example, as the resolution increases, the number of sections increases and the color of an actual tooth can be more accurately represented as data. Although there is no particular limitation on dividing the tooth to be measured into several sections, the sections can be divided in consideration of 3D printing. For example, considering that 3D printing is performed in a layered manner, the tooth to be measured may also be divided into a plurality of sections formed along the lateral direction of the tooth and stacked in the longitudinal direction. Alternatively, the tooth to be measured may be divided into several sections in a mosaic form.

The patient's tooth color data generated in the first step (S100) may further include data on the position of the tooth, patient information, information on tooth shape, etc., if necessary.

Next, a second step (S200) of setting tooth color data for each section of artificial tooth using the patient's tooth color data generated in the first step (S100) is performed.

In the second step (S200), the tooth color data for each section of the artificial tooth may have a structure similar to the patient's tooth color data generated in the first step (S100). In particular, having a similar data structure may mean using the same or similar color coordinate system. For example, if the patient's tooth color data conforms to the CIE L*a*b* coordinate system as described above and includes a* coordinate values representing color, b* coordinate values, and L* coordinate values representing brightness, the tooth color data for each section may also include a* coordinate values, b* coordinate values, and L* coordinate values. In addition, the tooth color data for each section may further include section data on the position within the tooth.

In the second step (S200), the section data included in the tooth color data for each section may include information about the position of the section, the size of the section, etc. The section data defined in the second step (S200) does not necessarily match the section information provided in the first step (S100). In some cases, the section data of the second step (S200) and the section data of the first step (S100) may be different. Specifically, if the section data in the first step (S100) can be determined according to the resolution for measuring the color of the patient's teeth, the section data in the second step (S200) may be divided in consideration of 3D printing. For example, considering that 3D printing is performed in a layered manner, the artificial tooth may be divided into a plurality of sections formed along the lateral direction of the tooth and stacked in the longitudinal direction. In some cases, tooth color data for each section may be generated for three regions by dividing the tooth into upper, middle, and lower regions.

In the second step (S200), the tooth color data for each section may represent a form in which one color coordinate value is assigned to each section. For example, the tooth color data for each section may be provided in a form in which values (a1, b1, L1) are assigned to a first section and values (a2, b2, L2) are assigned to a second section. In particular, the color coordinate values assigned to each section may be determined in consideration of color coordinate values according to patient's tooth color data corresponding to each section. For example, a plurality of patient's tooth color data may exist in a region of an actual tooth corresponding to a region represented by one section of tooth color data. In this case, one section of tooth color data may correspond to a plurality of patient's tooth color data. When a plurality of patient's tooth color data has different color coordinate values, one section of tooth color data corresponding thereto may be determined in consideration of different color coordinate values of the plurality of patient's tooth color data.

The second step (S200) may be performed in a form of dividing the color coordinate values consisting of (a, b, L) into a coordinate plane for (a, L) and a coordinate plane for (b, L), setting a section for color implementation on each coordinate plane, and dividing the corresponding section into equal parts according to the number of sections. The sections may be set to maximally include the color coordinate values according to the patient's tooth color data generated in the first step (S100). Hereinafter, more details on this will be described.

Next, in the third step (S300), a tooth image is implemented based on the tooth color data for each section determined in the second step (S200). The implemented tooth image may be displayed electronically. The user may adjust tooth color data for each section based on the tooth image implemented in the third step (S300). Alternatively, tooth color data for each section may also be adjusted according to a preset algorithm.

Next, in the fourth step (S400), artificial tooth color data is determined. The determined artificial tooth color data may be tagged and stored together with the information of tooth position, patient information, etc. The stored artificial tooth color data may be transferred to a 3D printing system for manufacturing artificial teeth.

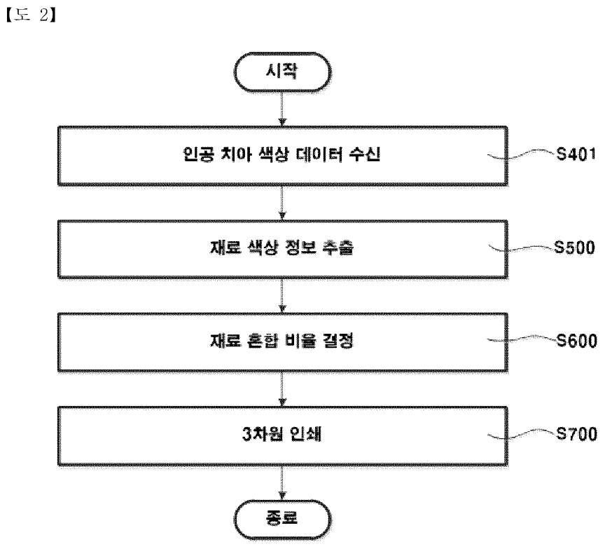

FIG. 2 is a flowchart showing a method for performing 3D printing after setting a customized tooth color according to one embodiment of the present invention.

With reference to FIG. 2, a step (S401) of receiving the artificial tooth color data determined in the preceding customized tooth color setting system is first performed. The step (S401) of receiving the artificial tooth color data may be performed through online communication or offline communication. For example, when the customized tooth color setting system and the artificial tooth manufacturing system are physically connected by wires, etc., artificial tooth color data may be transmitted offline. In contrast, when the customized tooth color setting system and the artificial tooth manufacturing system are not physically connected, artificial tooth color data may be wirelessly transmitted and received. However, the method for transmitting and receiving artificial tooth color data is not particularly limited.

Next, a step (S500) of extracting material color is performed. The step of extracting material color is to extract the color information of the material used to manufacture the artificial tooth. In particular, the color information conforms to the CIE L*a*b* coordinate system as reviewed above, and may include a* coordinate values representing color, b* coordinate values, and L* coordinate values representing brightness.

There may be at least two or more materials to be extracted in the step (S500) of extracting material color. Alternatively, at least two or more different materials may have different color information. For example, the first material may have color information closer to white, and the second material may have color information closer to yellow. By mixing two or more materials having different color information, a color according to the received artificial tooth color data may be implemented.

Next, a step (S600) of determining material mixing ratio may be performed. The material mixing ratio may vary according to the section set in the received artificial tooth color data. For example, when the artificial tooth color data divides the tooth into upper/middle/lower sections and includes color coordinate values for each section, the material mixing ratio may vary for each upper/middle/lower section. In particular, the material mixing ratio may be normalized using color coordinate values for each section of artificial tooth color data. For example, a straight-line mixture ratio graph drawn using the ratio (y) of two materials and the position (x) within the tooth may be normalized to have curves upward or downward along a straight line using the color coordinate values for each section of the artificial tooth color data. Accordingly, the ratio of the two materials does not change linearly according to the position within the tooth, but may change to satisfy an exponential or spline curve to fit the artificial tooth color data. Thus, manufactured artificial teeth can be manufactured to have a natural gradation close to actual teeth even using the two materials.

Next, a step of 3D printing (S700) is performed after determining the material mixing ratio according to the position (section) within the tooth. The method of 3D printing is not particularly limited. For example, various methods such as FDM, SLA, DLP, SLS, DOD, DMLS, SLM, EBM, etc. may be used in 3D printing.

The 3D printing (S700) may be performed in consideration of the material mixing ratio for each position (section) as determined above, and may be performed with reference to the information on tooth shape.

In the above, the method for setting a customized tooth color according to one embodiment of the present invention and the method for performing 3D printing after setting the customized tooth color have been examined. Hereinafter, a system for performing the methods will be examined.

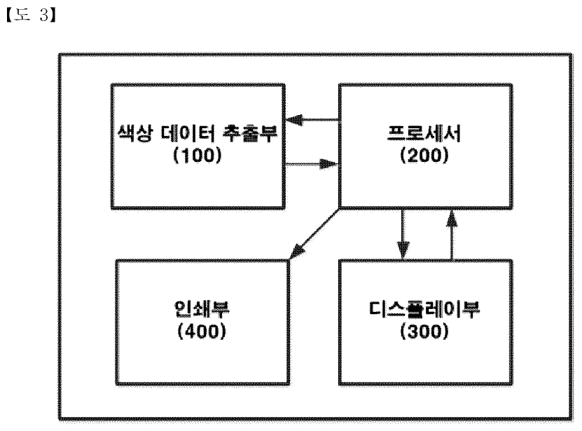

FIG. 3 is a block diagram showing a customized tooth color implementation system according to one embodiment of the present invention.

With reference to FIG. 3, the customized tooth color implementation system includes a color data extraction unit (100), a processor (200), a display unit (300), and a printing unit (400).

The color data extraction unit (100) generates the patient tooth color data described above. The color data extraction unit (100) may include an optical photographing device or a spectrophotometer. Alternatively, in some cases, the color data extraction unit (100) may extract color information of materials used to manufacture artificial teeth.

The processor (200) may set tooth color data for each section of an artificial tooth, correct errors, and determine the material mixing ratio. For this purpose, the processor (200) may receive patient's tooth color data from the color data extractor (100). Detailed operations for setting tooth color data for each section of the artificial tooth, correcting errors, and determining the material mixing ratio will be omitted to avoid duplication of content.

The display unit (300) outputs a simulation image produced according to tooth color data for each section of the artificial tooth. The display unit (300) may communicate with the processor (200) to receive a simulated image, and when a user modifies the simulated image, the processor may operate in a form of reflecting the modification and receiving the produced image again.

The printing unit (400) is a member for 3D printing an artificial tooth and may include a nozzle, etc. The shape of the printing unit (400) is not limited.

In the above, the method for setting a customized tooth color according to one embodiment of the present invention and the system for performing the same have been examined. Hereinafter, an actual customized tooth color setting and printing form will be examined.

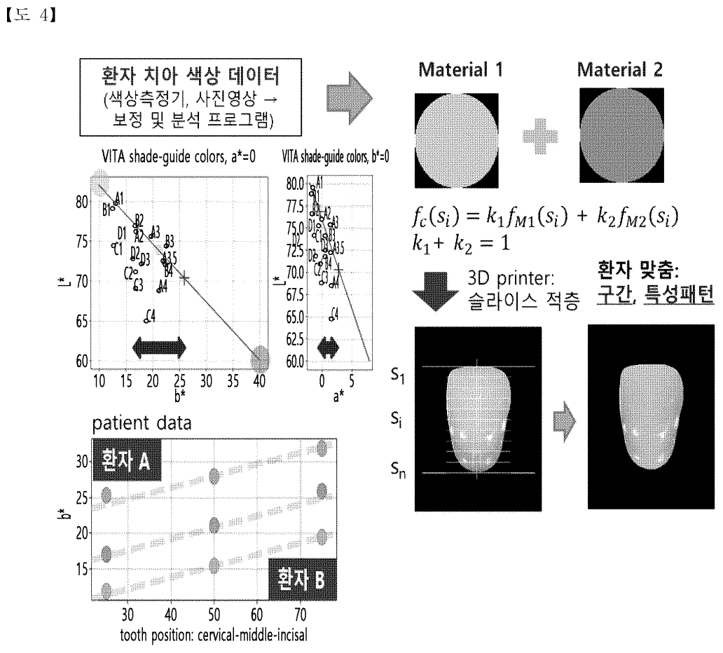

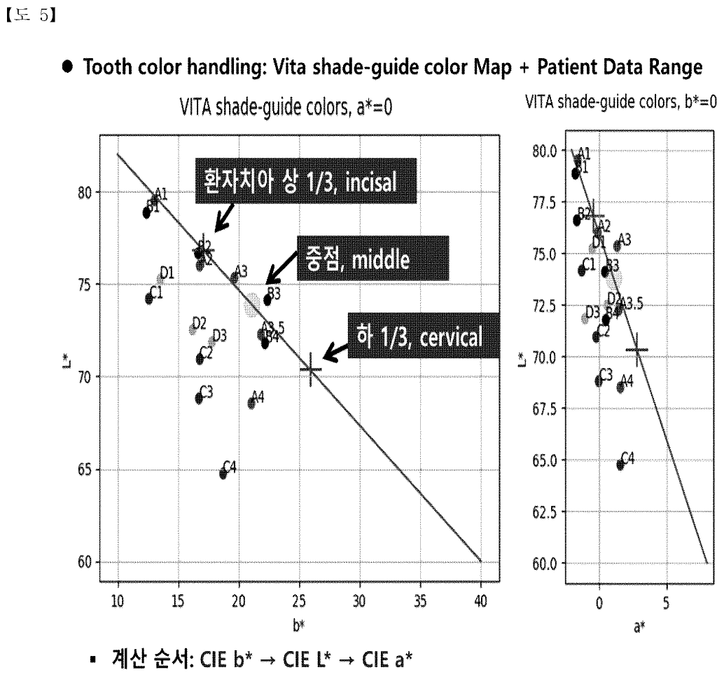

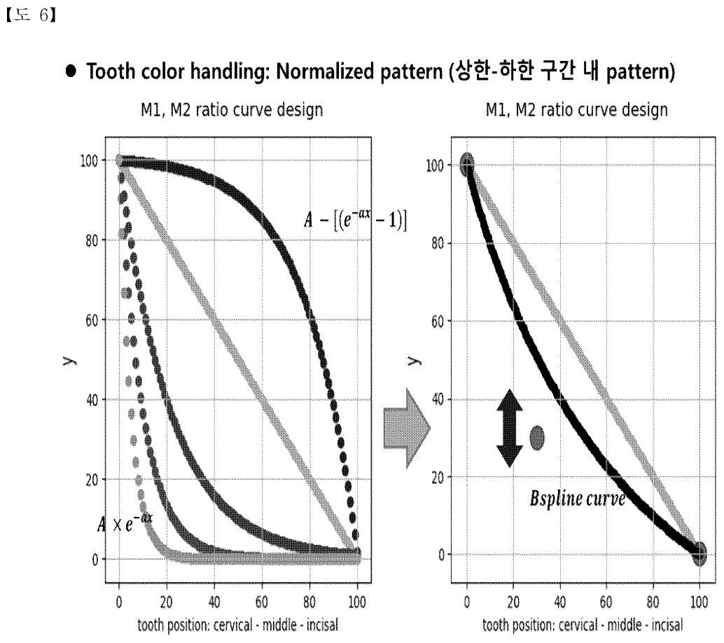

FIG. 4 is a graph and image data showing a color setting method according to one embodiment of the present invention. FIG. 5 is a graph showing a color setting method according to one embodiment of the present invention. FIG. 6 is a graph showing preparation steps for manufacturing artificial teeth according to one embodiment of the present invention.

First, with reference to FIG. 4, it can be seen that color coordinate values according to the Lightness-Chroma-hue (LCh) or the CIE L*a*b* coordinate system are extracted using patient's tooth color data and the color coordinate values are indicated on the (b, L) plane and the (a, L) plane. In addition, it can be seen that a color coordinate section (red arrow in the x-axis of the upper left graph) is set to include the color coordinate values displayed on the (b, L) plane and (a, L) plane. A mixing ratio of the first material (Material 1) and the second material (Material 2) is prepared so as to implement colors included in the set color coordinate section. The two materials are laminated or printed from S1 to Sn while varying the mixing ratio.

Next, with reference to FIG. 5, it can be seen that the generated patient's tooth color data is displayed on the (b, L) plane and the (a, L) plane. Here, an operation for deriving tooth color data for each section may be performed in the order of b values, L values, and a values.

Next, with reference to FIG. 6, it can be seen that y, which is the ratio of the first material (M1) to the second material (M2), is represented according to the position of the artificial tooth. Although the initial y value is set in the form of a straight line with respect to the position (x-axis) of the artificial tooth, the graph represented by the y values may be normalized to satisfy an exponential or spline curve using color coordinate values derived from patient's tooth color data as confirmed in FIG. 5. Accordingly, the y values are smoothly changed according to the position on the tooth, and the first material M1 and the second material M2 are combined in an appropriate ratio according to the y values, thereby generating an artificial tooth having a color similar to that of an actual tooth.

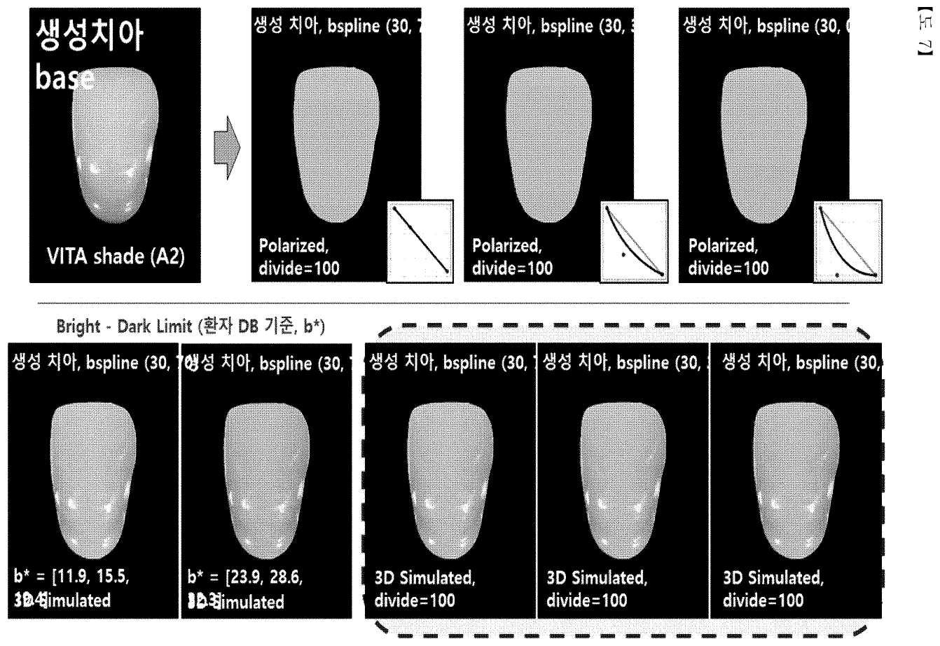

FIG. 7 is an image showing an artificial tooth manufactured according to one embodiment of the present invention. With reference to FIG. 7, images of artificial teeth manufactured by the same method as in FIGS. 4 to 6 can be seen. With reference to FIG. 7, the artificial tooth was divided into 100 sections and printed while varying the mixing ratio of the first material and the second material. In particular, it can be confirmed from the upper graph of FIG. 5 that the mixing ratio of the materials is not linear and changes to satisfy an exponential or spline curve. Accordingly, as shown below, it is possible to implement artificial teeth that are natural and close to actual teeth.

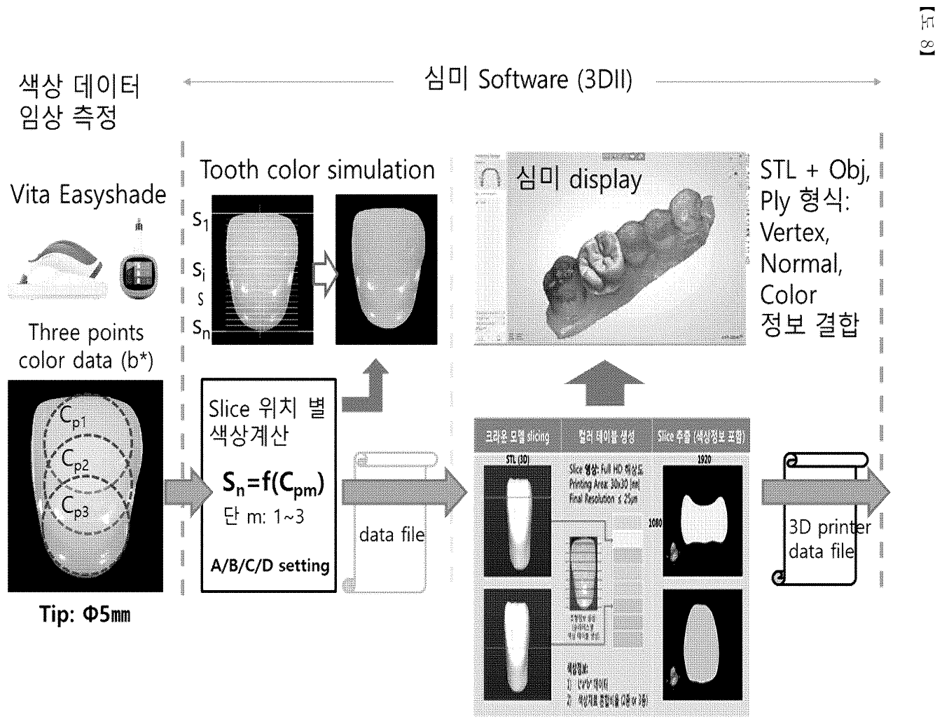

FIG. 8 is a flowchart showing in detail a method for setting a customized tooth color according to one embodiment of the present invention.

With reference to FIG. 8, patient's tooth color data is first generated in order to set a customized tooth color. In particular, a commercially available color measuring device such as Vita Easyshade may be used. As disclosed in the drawing (lower left part of the drawing), the tooth color may be performed by measuring the color in each of the first region (Cₚ₁), the second region (Cₚ₂), and the third region (Cₚ₃) sequentially provided from the root of the tooth along the height direction of the tooth. In this case, the first region (Cₚ₁), the second region (Cₚ₂), and the third region (Cₚ₃) may be circular, and the size of the regions may be about 5 mm in diameter. The first region (Cₚ₁), the second region (Cₚ₂), and the third region (Cₚ₃) may overlap in some regions as shown in the drawing. However, the above method is exemplary, and in some cases, the patient's tooth color data may be generated by measuring the color in an arbitrary region within the tooth and calculating color deviation from the root of the tooth along the height direction. Any of the above two methods may be selected in consideration of the size and shape of the measurement target. For example, when the tooth is small, a method of measuring color in one region and calculating color deviation may be used rather than measuring color in three regions.

Next, the second step of setting tooth color data for each section of the artificial tooth based on the patient's tooth color data is performed. As can be seen in the drawing, it may be an operation of calculating and matching color data for each of a plurality of slices divided according to the height from the root to the top of the tooth.

Next, after tooth color data is generated, an aesthetic prediction display is performed based on the tooth color data for each section of the artificial tooth, and errors are corrected by comparing the artificial tooth displayed by the aesthetic prediction display with the patient's tooth color data (bottom right of the drawing). With respect to the second step (upper right of the drawing), it can be seen that color information is combined in various formats (vertex, normal) and an aesthetic display is implemented.

After completing the fourth step of determining artificial tooth color data by reflecting error correction through a series of processes described above, 3D printing information capable of 3D printing actual teeth may be generated. The 3D printing information may include information for printing teeth with materials. Information for printing teeth with materials may refer to information about which material should be arranged in which layer and how. When the materials are two or more types of materials having different colors, colors generated as images may be implemented depending on which layer and in what ratio the different colors are mixed and provided. Accordingly, the 3D printing information may include information for determining a mixing ratio of at least two or more materials having different colors.

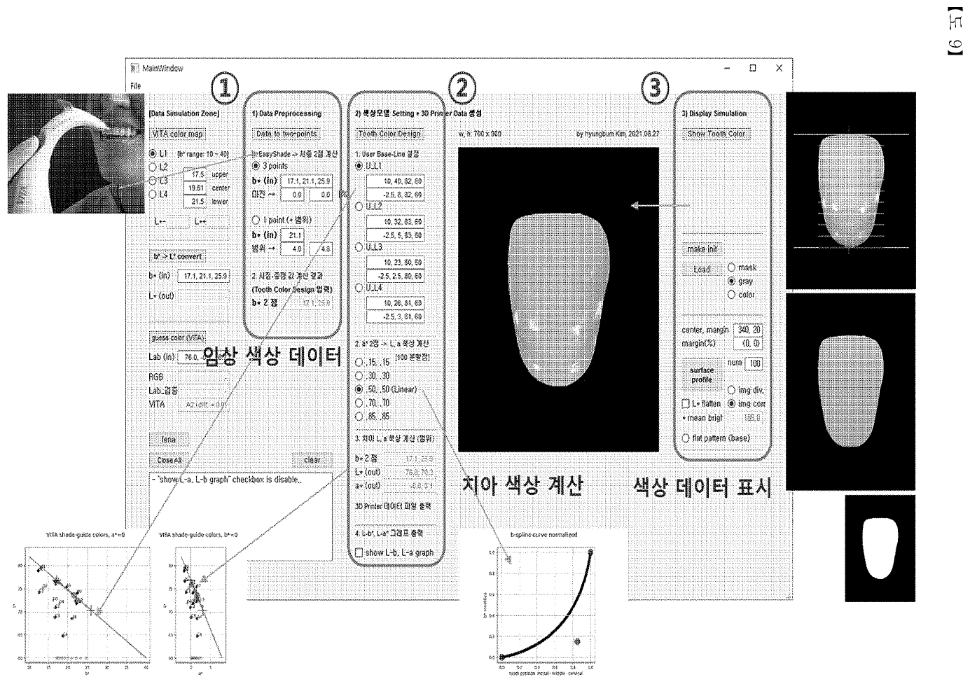

A specific embodiment of the method for setting a customized tooth color in the first to fourth steps described above can be examined in more detail in FIG. 9 below.

FIG. 9 is an actual implementation screen of a method for setting a customized tooth color according to one embodiment of the present invention.

With reference to FIG. 9, tooth color data received from the color measuring device is entered into the program as shown in \textcircled{1} in the drawing. With reference to part \textcircled{1} of the drawing, it can be seen that color data consists of b* values. In addition, a mode can be selected according to two cases: when colors are measured in three regions and when colors are measured in one region. When tooth color data is entered, the start value and end value of the b* value are calculated based on the input data (“2. Start-end point value calculation result” in region \textcircled{1} of the drawing).

Next, with reference to region \textcircled{2} of the drawing, tooth color data for each section of the artificial tooth is set based on the tooth color data. With reference to part \textcircled{2} of the drawing, it can be seen that L and a* values are calculated based on the start and end values of the received b* and b* values. The L and a* values may be calculated in a form in which a value that matches with the value of b* is loaded according to the preset model. For example, in the bottom left graph of the drawing, a graph model for the correlation between b* and L and the correlation between b* and a* is disclosed. L and a* can be calculated from b* value, a single variable, using the above graph. The above-described model may be a model calculated based on existing clinical data.

Next, with reference to region \textcircled{3} of the drawing, it can be confirmed that an aesthetic prediction display is performed. In particular, an operation of overlaying the model implemented by the previously calculated color data with glossiness may be performed. Specifically, as shown in the center of the drawing, an image providing an aesthetic feeling like an actual tooth can be generated by overlaying glossiness to the plain image on the right side of the drawing. The gloss data may be a preset gloss image layer, and the gloss image layer may be overlaid on the generated image.

As described above, according to the present invention, information for manufacturing an artificial tooth exhibiting aesthetic property similar to that of an actual tooth is automatically generated and provided even when only the b* value, which is a single variable, is measured and entered. In addition, a simulation is provided based on information for manufacturing an artificial tooth, and thus, the aesthetic feeling provided by a printed tooth may be readily predicted by a tooth manufacturer. Finally, since printing information for printing teeth reflecting the above-described information is generated, artificial teeth can be printed immediately by transmitting the printing information to 3D printing.

While the present invention has been described with reference to the preferred embodiments, it will be appreciated by those skilled in the corresponding art or those having ordinary knowledge in the corresponding art that the present invention may be modified and altered in various manners without departing from the spirit and technical scope of the present invention that are set forth in the following claims.

Therefore, the technical scope of the present invention is defined by the appended claims rather than the detailed description.

1-11. (canceled)

12. A method for setting a customized tooth color, comprising:
(1) generating a patient's tooth color data;
(2) setting tooth color data for each section of artificial tooth based on the patient's tooth color data;
(3) performing an aesthetic prediction display based on the tooth color data for each section of artificial tooth, and correcting errors by comparing the artificial tooth displayed by the aesthetic prediction display with the patient's tooth color data; and
(4) determining artificial tooth color data by reflecting error correction.

13. The method of claim 12, wherein the step (1) comprises generating the patient's tooth color data by measuring colors in each of a first region, a second region, and a third region sequentially provided from a root of the tooth along a height direction of the tooth.

14. The method of claim 12, wherein the step (1) comprises generating the patient's tooth color data by measuring color in an arbitrary region within the tooth and calculating color deviation from a root of the tooth along a height direction.

15. The method of claim 13, wherein the tooth color data is represented by the b* value of the CIE L*a*b* coordinate system.

16. The method of claim 14, wherein the tooth color data is represented by the b* value of the CIE L*a*b* coordinate system.

17. The method of claim 15, wherein the step (2) calculates L and a* values based on the b* value of the patient's tooth color data received in the step (1) and a preset tooth image model to generate tooth color data for each section of the artificial tooth.

18. The method of claim 17, wherein the tooth color data for each section of the artificial tooth comprises section data each consisting of the start and end values of L and a*.

19. The method of claim 12, wherein the aesthetic prediction display in the step (3) shows a colored tooth image based on the tooth color data for each section of the artificial tooth, and the tooth image is overlaid with a glossy image.

20. The method of claim 12, further comprising:
(5) calculating a mixing ratio of materials for printing an artificial tooth after the step (4).

21. The method of claim 20, wherein the step (5) determines the mixing ratio of at least two or more materials having different colors.

22. A computer program stored on a recording medium that configured to be interlocked with a computer device such that a method for setting a customized tooth color can be performed, wherein the method comprises:
(1) generating a patient's tooth color data;
(2) setting tooth color data for each section of artificial tooth based on the patient's tooth color data;
(3) performing an aesthetic prediction display based on the tooth color data for each section of artificial tooth, and correcting errors by comparing the artificial tooth displayed by the aesthetic prediction display with the patient's tooth color data; and
(4) determining artificial tooth color data by reflecting error correction.

23. A method for printing an artificial tooth, comprising:
three-dimensionally printing an artificial tooth using at least two or more materials having different colors; and
performing a method for setting a customized tooth color in order to determine the mixing ratio of the at least two or more materials, wherein the method comprises:
(1) generating a patient's tooth color data;
(2) setting tooth color data for each section of artificial tooth based on the patient's tooth color data;
(3) performing an aesthetic prediction display based on the tooth color data for each section of artificial tooth, and correcting errors by comparing the artificial tooth displayed by the aesthetic prediction display with the patient's tooth color data; and
(4) determining artificial tooth color data by reflecting error correction.
